SEQUENCE LISTING (6 sequences)

SEQ ID NO: 1 - protein, 621 aa, HCV
MSYYHHHHHHDYDIPTTENLYFQGAMDPEFSMSYTWTGALITPCAAEESQLPINALSNSLVRHRNMVYSTTSRSAALRQKKVTFDRLQVLDDHYRDVLKEMKAKASTVKAKLLSVEEACKLTPPHSAKSKFGYGAKDVRNLSSKAVDHIRSVWKDLLEDTETPIDTTIMAKNEVFCVQPEKGGRKPARLIVFPDLGVRVCEKMALYDVVSTLPQAVMGSSYGFQYSPKQRVEFLVNAWKSKKCPMGFSYDTRCFDSTVTESDIRVEESIYQCCDLAPEARQAIKSLTERLYIGGPLTNSKGQNCGYRRCRASGVLTTSCGNTLTCYLKASAACRAAKLQDCTMLVNGDDLVVICESAGTQEDAANLRVFTEAMTRYSAPPGDLPQPEYDLELITSCSSNVSVAHDASGKRVYYLTRDPTTPLARAAWETARHTPINSWLGNIIMYAPTLWARMVLMTHFFSILLAQEQLEKALDCQIYGACYSIEPLDLPQIIERLHGLSAFSLHSYSPGEINRVASCLRKLGVPPLRVWRHRARSVRAKLLSQGGRAATCGKYLFNWAVRTKLKLTPIPAASRLDLSGWFVAGYNGGDIYHSLSRARPRWFMLCLLLLSVGVGIYLLPNR

SEQ ID NO: 2 - protein, 591 aa, HCV
MGSSHHHHHHSSGLVPRGSHMSMSYTWTGALITPCAAEESQLPINALSNSLVRHRNMVYSTTSRSAALRQKKVTFDRLQVLDDHYRDVLKEMKAKASTVKAKLLSVEEACKLTPPHSAKSKFGYGAKDVRNLSSKAVDHIRSVWKDLLEDTETPIDTTIMAKNEVFCVQPEKGGRKPARLIVFPDLGVRVCEKMALYDVVSTLPQAVMGSSYGFQYSPKQRVEFLVNAWKSKKCPMGFSYDTRCFDSTVTESDIRVEESIYQCCDLAPEARQAIKSLTERLYIGGPLTNSKGQNCGYRRCRASGVLTTSCGNTLTCYLKASAACRAAKLQDCTMLVNGDDLVVICESAGTQEDAANLRVFTEAMTRYSAPPGDLPQPEYDLELITSCSSNVSVAHDASGKRVYYLTRDPTTPLARAAWETARHTPINSWLGNIIMYAPTLWARMVLMTHFFSILLAQEQLEKALDCQIYGACYSIEPLDLPQIIERLHGLSAFSLHSYSPGEINRVASCLRKLGVPPLRVWRHRARSVRAKLLSQGGRAATCGKYLFNWAVRTKLKLTPIPAASRLDLSGWFVAGYNGGDIYHSLSRARPR

SEQ ID NO: 3 - protein, 578 aa, HCV
SMSYTWTGALITPCAAEESQLPINALSNSLVRHRNMVYSTTSRSAALRQKKVTFDRLQVLDDHYRDVLKEMKAKASTVKAKLLSVEEACKLTPPHSAKSKFGYGAKDVRNLSSKAVDHIRSVWKDLLEDTETPIDTTIMAKNEVFCVQPEKGGRKPARLIVFPDLGVRVCEKMALYDVVSTLPQAVMGSSYGFQYSPKQRVEFLVNAWKSKKCPMGFSYDTRCFDSTVTESDIRVEESIYQCCDLAPEARQAIKSLTERLYIGGPLTNSKGQNCGYRRCRASGVLTTSCGNTLTCYLKASAACRAAKLQDCTMLVNGDDLVVICESAGTQEDAANLRVFTEAMTRYSAPPGDLPQPEYDLELITSCSSNVSVAHDASGKRVYYLTRDPTTPLARAAWETARHTPINSWLGNIIMYAPTLWARMVLMTHFFSILLAQEQLEKALDCQIYGACYSIEPLDLPQIIERLHGLSAFSLHSYSPGEINRVASCLRKLGVPPLRVWRHRARSVRAKLLSQGGRAATCGKYLFNWAVRTKLKLTPIPAASRLDLSGWFVAGYNGGDIYHSLSRARPRLEHHHHHH

SEQ ID NO: 4 - protein, 545 aa, HCV
SMSYTWTGALITPCAAEESQLPINALSNSLVRHRNMVYSTTSRSAALRQKKVTFDRLQVLDDHYRDVLKEMKAKASTVKAKLLSVEEACKLTPPHSAKSKFGYGAKDVRNLSSKAVDHIRSVWKDLLEDTETPIDTTIMAKNEVFCVQPEKGGRKPARLIVFPDLGVRVCEKMALYDVVSTLPQAVMGSSYGFQYSPKQRVEFLVNAWKSKKCPMGFSYDTRCFDSTVTESDIRVEESIYQCCDLAPEARQAIKSLTERLYIGGPLTNSKGQNCGYRRCRASGVLTTSCGNTLTCYLKASAACRAAKLQDCTMLVNGDDLVVICESAGTQEDAANLRVFTEAMTRYSAPPGDLPQPEYDLELITSCSSNVSVAHDASGKRVYYLTRDPTTPLARAAWETARHTPINSWLGNIIMYAPTLWARMVLMTHFFSILLAQEQLEKALDCQIYGACYSIEPLDLPQIIERLHGLSAFSLHSYSPGEINRVASCLRKLGVPPLRVWRHRARSVRAKLLSQGGRAATCGKYLFNWAVRTKLAAALEHHHHHH

SEQ ID NO: 5 - protein, 591 aa, HCV
SMSYTWTGALITPCAAEESQLPINALSNSLVRHRNMVYSTTSRSAALRQKKVTFDRLQVLDDHYRDVLKEMKAKASTVKAKLLSVEEACKLTPPHSAKSKFGYGAKDVRNLSSKAVDHIRSVWKDLLEDTETPIDTTIMAKNEVFCVQPEKGGRKPARLIVFPDLGVRVCEKMALYDVVSTLPQAVMGSSYGFQYSPKQRVEFLVNAWKSKKCPMGFSYDTRCFDSTVTESDIRVEESIYQCCDLAPEARQAIKSLTERLYIGGPLTNSKGQNCGYRRCRASGVLTTSCGNTLTCYLKASAACRAAKLQDCTMLVNGDDLVVICESAGTQEDAANLRVFTEAMTRYSAPPGDLPQPEYDLELITSCSSNVSVAHDASGKRVYYLTRDPTTPLARAAWETARHTPINSWLGNIIMYAPTLWARMVLMTHFFSILLAQEQLEKALDCQIYGACYSIEPLDLPQIIERLHGLSAFSLHSYSPGEINRVASCLRKLGVPPLRVWRHRARSVRAKLLSQGGRAATCGKYLFNWAVRTKLKLTPIPAASRLDLSGWFVAGYNGGDIYHSLSRARPRWFMLCLLLLSVGVGIYLLPNR

SEQ ID NO: 6 - protein, 627 aa, HCV
MSYYHHHHHHDYDIPTTENLYFQGAMDPEFEDVVCCSMSYTWTGALITPCAAEESQLPINALSNSLVRHRNMVYSTTSRSAALRQKKVTFDRLQVLDDHYRDVLKEMKAKASTVKAKLLSVEEACKLTPPHSAKSKFGYGAKDVRNLSSKAVDHIRSVWKDLLEDTETPIDTTIMAKNEVFCVQPEKGGRKPARLIVFPDLGVRVCEKMALYDVVSTLPQAVMGSSYGFQYSPKQRVEFLVNAWKSKKCPMGFSYDTRCFDSTVTESDIRVEESIYQCCDLAPEARQAIKSLTERLYIGGPLTNSKGQNCGYRRCRASGVLTTSCGNTLTCYLKASAACRAAKLQDCTMLVNGDDLVVICESAGTQEDAANLRVFTEAMTRYSAPPGDLPQPEYDLELITSCSSNVSVAHDASGKRVYYLTRDPTTPLARAAWETARHTPINSWLGNIIMYAPTLWARMVLMTHFFSILLAQEQLEKALDCQIYGACYSIEPLDLPQIIERLHGLSAFSLHSYSPGEINRVASCLRKLGVPPLRVWRHRARSVRAKLLSQGGRAATCGKYLFNWAVRTKLKLTPIPAASRLDLSGWFVAGYNGGDIYHSLSRARPRWFMLCLLLLSVGVGIYLLPNR

(19) United States
(12) Patent Application Publication
(10) Pub. No.: US 2004/0110126 A1
(43) Pub. Date: Jun. 10, 2004

(54) Hcv polymerase inhibitor assay

(76) Inventors: George Kukolj, Mont-Royal (CA); Ginette McKercher, Montreal (CA)

Correspondence Address: BOEHRINGER INGELHEIM CORPORATION, 900 RIDGEBURY RD, P O BOX 368, RIDGEFIELD, CT, 06877, US

(21) Appl. No.: 10/471,164

(22) PCT Filed: Mar. 6, 2002
(86) PCT No.: PCT/CA02/00323

(51) Int. Cl. C12Q 1/70; C12P 19/34

(52) U.S. Cl. 435/5; 435/91.2

(57) ABSTRACT

The present invention provides a novel method for the identification of inhibitors of HCV NS5B polymerase that uses an HCV NS5B RNA-dependent RNA polymerase having a higher Km than the full length native NS5B polymerase to ensure identification of inhibitors of HCV polymerase primer-template binding, including moderately inhibitory compounds. The method includes providing a decreased-affinity HCV NS5B polymerase, an appropriate primer-template, and a plurality of appropriate ribonucleotide triphosphates, incubating the HCV NS5B polymerase with the primer-template in the presence and absence of a potential inhibitor, measuring the presence of any polymerase products formed in the presence and absence of the potential inhibitor; and comparing the amount of the polymerase products formed in the presence and absence of the potential inhibitor; wherein a decrease in the amount of the polymerase products formed in the presence of the potential inhibitor compared to the amount of polymerase products formed in the absence of the potential inhibitor is indicative of a potential primer-template binding inhibitor of HCV NS5B RNA-dependent RNA polymerase.

### FIELD OF THE INVENTION

The present invention relates generally to a HCV RNA-dependent RNA polymerase with a low affinity for RNA primer-template. Particularly, the invention relates to a HCV NS5B polymerase with a Km higher than native HCV NS5B RNA-dependent RNA polymerase. More particularly, the invention relates to the use of such a NS5B polymerase for the identification of inhibitors of NS5B activity, particularly inhibitors of NS5B primer-template binding.

### BACKGROUND

Hepatitis C virus (HCV) is the major etiological agent of post-transfusion and community-acquired non-A non-B hepatitis worldwide. It is estimated that over 200 million people worldwide are infected by the virus. A high percentage of carriers become chronically infected and many progress to chronic liver disease, so called chronic hepatitis C. This group is in turn at high risk for serious liver disease such as liver cirrhosis, hepatocellular carcinoma and terminal liver disease leading to death.

The mechanism by which HCV establishes viral persistence and causes a high rate of chronic liver disease has not been thoroughly elucidated. It is not known how HCV interacts with and evades the host immune system. In addition, the roles of cellular and humoral immune responses in protection against HCV infection and disease have yet to be established.

HCV is an enveloped positive strand RNA virus in the Flaviviridae family. The single strand HCV RNA genome is of positive polarity and comprises one open reading frame (ORF) of approximately 9600 nucleotides in length, which encodes a linear polyprotein of approx. 3010 amino acids. In infected cells, this polyprotein is cleaved at multiple sites by cellular and viral proteases to produce structural and non-structural (NS) proteins. The structural proteins (C, E1, E2 and E2-p7) comprise polypeptides that constitute the virus particle (Hijikata et al., 1991; Grakoui et al., 1993(a)). The non-structural proteins (NS2, NS3, NS4A, NS4B, NS5A, NS5B) encode for enzymes or accessory factors that catalyze and regulate the replication of the HCV RNA genome. Processing of the structural proteins is catalyzed by host cell proteases (Hijikata et al., 1991). The generation of the mature non-structural proteins is catalyzed by two virally encoded proteases. The first is the NS2/3 zinc-dependent metalloprotease which auto-catalyses the release of the NS3 protein from the polyprotein. The released NS3 contains a N-terminal serine protease domain (Grakoui et al., 1993(b); Hijikata et al., 1993) and catalyzes the remaining cleavages from the polyprotein. The released NS4A protein has at least two roles. The first role is forming a stable complex with NS3 protein and assisting in the membrane localization of the NS3/NS4A complex (Kim et al., 1999); the second is acting as a cofactor for NS3 protease activity. This membrane-associated complex, in turn catalyzes the cleavage of the remaining sites on the polyprotein, thus effecting the release of NS4B, NS5A and NS5B (Bartenschlager et al., 1993; Grakoui et al., 1993(a); Hijikata et al, 1993; Love et al., 1996; reviewed in Kwong et al., 1998). The C-terminal segment of the NS3 protein also harbors nucleoside triphosphatase and RNA helicase activity (Kim et al., 1995). The function of the protein NS4B is unknown. NS5A, a highly phosphorylated protein, seems to be responsible for the Interferon resistance of various HCV genotypes (Gale Jr. et al. 1997; Reed et al., 1997). NS5B is an RNA-dependent RNA polymerase (RdRp) that is involved in the replication of HCV (Behrens et al., 1996).

The cloned and characterized partial and complete sequences of the HCV genome have been analyzed with regard to appropriate targets for a prospective antiviral therapy. The following four viral enzyme activities provide possible targets: (1) the NS2/3 protease; (2) the NS3/4A protease complex; (3) the NS3 Helicase; and (4) the NS5B RNA-dependent RNA polymerase (NS5B RdRp). The NS5B RNA-dependent RNA polymerase has been crystallized to reveal a structure reminiscent of other nucleic acid polymerases (Bressanelli et al. 1999; Ago et al. 1999; Lesburg et al. 1999).

The HCV NS5B polymerase is a prime target in the search for inhibitors of HCV replication. It has been recently demonstrated that mutations destroying NS5B activity abolish infectivity of RNA in a chimp model (Kolykhalov, 2000). The initial step of viral RNA replication is recognition of the 3′-end of RNA template by NS5B (RdRp), which may occur directly or indirectly with the help of cellular proteins (Lai, 1998; Strauss et al., 1999). HCV polymerase then proceeds to elongate this template and form double stranded RNA. Inhibitors of the HCV polymerase can therefore interfere at two separate steps during the RNA replication, i.e. 1) primer-template binding and 2) elongation.

A variety of in vitro assays for HCV NS5B polymerase activity have been developed. Commonly, the standard reaction mixture generally consists of buffers, salts, divalent cations, reducing agents, as well as nucleoside triphosphates and an RNA template and primer. Most of these assays utilize synthetic homopolymeric templates/primers (Behrens et al., 1996; Yuan et al., 1997; Lohmann et al., 1997 and 1998; Yamashita et al., 1998; Ferrari et al., 1999; Oh et al., 1999; Ishii et al., 1999; Tomei et al, 2000; Johnson et al, 2000; Qin et al., 2001 and 2002; Hagedorn et al., WO 97/12033, U.S. Pat. No. 5,981,247, WO 00/06529 by Instituto di Ricerche di Biologia Moleculare P. Angeletti S.P.A, WO 99/51781 and WO 00/13708 by Viropharma Inc. WO 01/47883 by Japan Tobacco Inc. reports a series of compounds having inhibitory activity against HCV NS5B polymerase, and report assays for measuring HCV polymerase inhibitory activity.

The recombinant HCV NS5B polymerase enzyme used to perform assays in the prior art is predominantly produced and isolated from E.coli or baculovirus-infected insect cells (such as Sf9). Expression of the full-length HCV NS5B, either untagged or tagged (with a hexa-His-tag or GST-tag), results in insoluble protein requiring extraction with detergents (such as Triton X-100, NP40 and/or CHAPS), salt and glycerol (Behrens et al., 1996; Lohmann et al., 1997 and 1998; Oh et al., 1999; Ishii et al., 1999; Tomei et al, 2000; Johnson et al, 2000; Qin et al., 2001 and 2002). The HCV NS5B protein has a highly conserved C-terminal hydrophobic segment and truncation of this C-terminal portion in recombinant clones has allowed for the expression and isolation of a soluble form of the enzyme (Yamashita et al., 1998; Ferrari et al., 1999; Tomei et al, 2000; Del Vecchio WO 99/29843).

The activity of NS5B in a common in vitro polymerase reaction with homopolymeric RNA requires interaction with multiple substrates that include a primer-template and a ribonucleotide triphosphate. Steady-state kinetic parameters, such as the Kₘ, can be determined for both the primer-template and the ribonucleotide triphosphate substrates (Ferrari et al., 1999). Recombinant HCV polymerases disclosed in the prior art have high affinity (low Kₘ value) for primer-templates, and the use of native NS5B in assays to identify inhibitors is similarly problematic in that native NS5B has high affinity for primer-templates. In order to identify test compounds that inhibit polymerases of high affinity (low Kₘ), it is important that the test compound be present in concentration below or near to the Kₘ for the primer-template in the inhibition assay in order for the inhibitor to compete with the substrates of the polymerase reaction.

Currently existing HCV NS5B assays that utilize recombinant or native HCV polymerases to identify potential inhibitors of native NS5B RNA-dependent RNA polymerase encoded by the HCV RNA genome can identify inhibitors of RNA-binding or ribonucleotide triphosphate incorporation. Inhibitory compounds that compete with these substrates must have affinities comparable to (or greater than) that of the primer-template or NTP for the polymerases. Using the prior art screening assays to screen a library of compounds restricts identification of competitive inhibitors of primer-template binding to those with high affinity and would not identify inhibitors with moderate or low affinity.

The development of new and specific anti-HCV treatments is a high priority, and virus-specific functions essential for replication are the most attractive targets for drug development. The absence of RNA-dependent RNA polymerases in mammals, and the fact that this enzyme appears to be essential to viral replication, would suggest that the HCV NS5B polymerase is an ideal target for anti-HCV therapeutics.

There thus remains an unmet need for the development of a method with improved sensitivity and greater dynamic range for identifying test compounds with moderate or low affinity that are capable of modulating, particularly inhibiting, HCV NS5B primer-template binding activity. Such compounds would serve as an ideal starting point for further medicinal chemistry optimization of an anti-HCV therapeutic.

### SUMMARY OF THE INVENTION

The present invention reduces the difficulties and disadvantages of the prior art by providing a novel method for the identification of inhibitors of HCV NS5B polymerase that uses an HCV NS5B RNA-dependent RNA polymerase having a higher Kₘ than the native NS5B polymerase to ensure identification of inhibitors of HCV polymerase primer-template binding. In particular, the present invention concerns the design of a recombinant HCV NS5B construct that has a higher Kₘ for RNA (primer-template) and thereby a wider range of sensitivities for identifying test compounds capable of modulating (particularly inhibiting) HCV NS5B activity.

Therefore, in a first embodiment of the present invention, there is provided a method for identifying a potential inhibitor of the binding between a HCV NS5B RNA-dependent RNA polymerase and an appropriate primer-template, the method comprising:

a) providing a HCV NS5B polymerase, an appropriate primer-template, and a plurality of appropriate ribonucleotide triphosphates, wherein the HCV NS5B polymerase has an affinity for the primer-template that is decreased relative to that of native HCV NS5B RNA-dependent RNA polymerase;

b) incubating the HCV NS5B polymerase with the primer-template in the presence and absence of a potential inhibitor;

c) measuring the presence of any polymerase product formed upon binding of the HCV NS5B polymerase and subsequent elongation of the primer upon incorporation of one or more ribonucleotide triphosphates as ribonucleotide monophosphates into the primer in the presence and absence of the potential inhibitor; and

d) comparing the amount of the polymerase products formed in the presence and absence of the potential inhibitor;

wherein a decrease in the amount of the polymerase products formed in the presence of the potential inhibitor compared to the amount of polymerase products formed in the absence of the potential inhibitor is indicative of a potential primer-template binding inhibitor of HCV NS5B RNA-dependent RNA polymerase.

In a second embodiment of the invention, there is provided a NS5B polymerase enzyme that has a low affinity towards its primer-template. Particularly, the present invention provides a hepatitis C virus RNA-dependent RNA polymerase that has a Kₘ of above 10 nM towards poly(A)/oligo(U).

In a third embodiment of the present invention, there is provided a kit for identifying a test compound as an inhibitor of the binding between an HCV NS5B polymerase and an appropriate primer-template, the kit comprising:

(a) a first reagent comprising an HCV NS5B polymerase, wherein the HCV NS5B polymerase has an affinity for the primer-template that is decreased relative to that of a native HCV NS5B polymerase;

(b) a second reagent comprising an appropriate primer-template capable of being bound by the HCV NS5B polymerase in the absence of the test compound, wherein the primer is affinity-tagged at its 5'C position;

(c) a third reagent comprising a plurality of appropriate radio-labeled [5,6 ³H]-ribonucleotide triphosphates capable of being incorporated as radio-labeled [5,6 ³H]-ribonucleotide monophosphates into the primer upon binding of the HCV NS5B polymerase and subsequent elongation of the primer, thereby forming polymerase products; and

(d) a fourth reagent comprising a plurality of receptor-coated solid support suitable to capture the affinity-tagged primer-template and any formed affinity-tagged polymerase products, whereby, upon measurement, the intensity of signal emitted from the solid support is proportional to the level of formation of radio-labeled polymerase products.

In a fourth embodiment, the present invention encompasses the use of the method to identify a potential inhibitor of native HCV NS5B RNA-dependent RNA polymerase and contacting the native HCV NS5B RNA-dependent RNA polymerase with the test compound identified as having potential inhibitory activity in step (a), whereby polymerase activity of the native HCV NS5B RNA-dependent RNA polymerase is inhibited.

The advantages of the invention are manyfold. The present invention provides an assay that is easy to perform on large libraries of compounds, and has improved sensitivity for detecting inhibitors that would not be identified as such using native NS5B polymerase. Importantly, this assay teaches the importance of utilizing a polymerase NS5B construct that has a higher Kₘ than that of the native NS5B polymerase so that compounds are identified that can act competitively towards one or both of the main substrates of the reaction. Use of polymerase constructs having a lower affinity (higher Kₘ) towards the primer-template than that of native NS5B polymerase is particularly useful for identifying potential inhibitors in screening large libraries of compounds.

Other objects, advantages and features of the present invention will become more apparent upon reading of the following non-restrictive description of the preferred embodiments with reference to the accompanying drawings which are exemplary and should not be interpreted as limiting the scope of the invention.

### BRIEF DESCRIPTION OF THE DRAWINGS

Having thus generally described the invention, reference will now be made to the accompanying drawings, showing by way of illustration a preferred embodiment thereof, and in which:

FIG. 1 shows the effect of primer length on HT-NS5B enzymatic activity using Oligos U₄, U₆, U₈, U₁₀, U₁₂, U₁₄, U₁₆ and U₁₈;

FIG. 2A shows the determination of the mode of inhibition and constant of inhibition (Kᵢ) towards a primer-template with a representative test compound for HT-NS5B using a Dixon plot;

FIG. 2B shows the determination of the mode of inhibition and constant of inhibition (Kᵢ) towards a primer-template with a representative compound for HT-NS5B using a Cornish-Bowden plot;

FIG. 3A shows the determination of the mode of inhibition and constant of inhibition (Kᵢ) towards UTP with a representative compound for HT-NS5B using a Dixon plot;

FIG. 3B shows the determination of the mode of inhibition and constant of inhibition (Kᵢ) towards UTP with a representative compound for HT-NS5B using a Cornish-Bowden plot.

### DETAILED DESCRIPTION OF THE INVENTION

Definitions

Unless defined otherwise, the scientific and technological terms and nomenclature used herein have the same meaning as commonly understood by a person of skill in the art to which this invention pertains but should not be interpreted as limiting the scope of the present invention.

Amino acid residues described herein are preferred to be in the “L” isomeric form. However, residues in the “D” isomeric form may be substituted for any L-amino acid residue, provided the desired properties of the polypeptide are retained.

All amino-acid residue sequences represented herein conform to the conventional left-to-right amino-terminus to carboxy-terminus orientation in accordance with the recommendations of the IUPAC-IUB Biochemical Nomenclature Commission (1972).

Nucleotide sequences are presented herein by single strand, in the 5′ to 3′ direction, from left to right, using the one letter nucleotide symbols as commonly used in the art and according to IUPAC.

Conventional methods of gene isolation, molecular cloning, vector construction, etc., are well known in the art and summarized, for example, in Sambrook, J. et al., Molecular Cloning: A laboratory Manual, Second Edition, Cold Spring Harbor Laboratory Press, Cold Spring Harbor, N.Y., and Ausubel et al., Current Protocols in Molecular Biology, John Wiley & Sons, Inc. One skilled in the art can readily reproduce the plasmid vectors described herein without undue experimentation employing these methods. The various nucleic acid sequences, fragments, etc., necessary for this and other purposes can be readily obtained as components of commercially available plasmids, or are otherwise well known in the art and publicly available, or readily reproducible based upon published information.

The term “affinity tag”, as used herein, means a ligand (that is linked preferably to a primer-template) whose strong affinity for a “receptor” can be used to extract from a solution the entity to which the ligand is attached. Examples of such ligands include biotin or a derivative thereof, a histidine polypeptide, an amylose sugar moiety or a defined epitope recognizable by a specific antibody. Such “affinity tags” are preferably attached to the primer-template in solution and is captured by a suitable “receptor”moiety attached to a solid support.

A “derivative” of the HCV NS5B polypeptide or a fragment thereof means a polypeptide modified by varying the amino acid sequence of the protein, e.g. by manipulation of the nucleic acid encoding the protein or by altering the protein itself. Such derivative of the natural amino acid sequence may involve insertion, addition, deletion or substitution of one or more amino acids, and may or may not alter the essential activity of the original HCV NS5B polypeptide. As mentioned above, the HCV NS5B polypeptide or protein of the invention includes any analogue, fragment, derivative or mutant which is derived from a HCV NS5B polypeptide and which retains at least one property or other characteristic of the HCV NS5B polypeptide.

The terms “elongation” or “extension” are used interchangeably and mean the consecutive addition of nucleotides as directed by a complementary template of DNA or RNA that is carried out by an appropriate polymerase. In the particular context of this invention, elongation or extension is carried out on an RNA template by a flavivirus RNA-dependent RNA polymerase, particularly the HCV NS5B RdRp.

A “fragment” or “portion” of the HCV NS5B polypeptide means a stretch of amino acid residues of sufficient length or an NS5B polypeptide having amino acids deleted therein, while retaining at least one of its function such as binding to a template, priming, or elongation along a template.

The term “initiation” refers the first step of RNA synthesis, that incorporates the initial 5′ position nucleotide of the nascent RNA chain. This reaction is also referred to as “priming”.

The term “NS5B” refers to a portion of the HCV genome located near the 3′ end of the viral genome that specifies the region encoding a protein, termed the “NS5B protein”, “NS5B polypeptide”, “NS5B polymerase” or combinations of these terms which are used interchangeably herein. NS5B in its natural state, functions as an RNA-dependent RNA polymerase (RdRp). The nucleic acid region encoding the NS5B protein may also be referred to as the “NS5B gene”. Thus, the term “NS5B” may refer to either a nucleic acid encoding the NS5B polypeptide, to an NS5B gene or to an NS5B polypeptide, or to any portions thereof, depending on the context in which the term is used. NS5B may further refer to natural allelic variants, mutants and derivatives of either NS5B nucleic acid sequences or NS5B polypeptides. The NS5B nucleic acid, NS5B gene or NS5B protein referred to is a functional polymerase, or to a non-functional polymerase that still binds to an appropriate template.

The term “plasmid” refers to an extrachromosomal genetic element. The starting plasmids herein are either commercially available, publicly available on an unrestricted basis, or can be constructed from available plasmids in accordance with published procedures. In addition, equivalent plasmids to those described are known in the art and will be apparent to the ordinarily skilled artisan.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be elongated (extended) at its 3′ terminus by the addition of nucleotides by the action of a polymerase or similar activity to yield a primer elongation (extension) product. The primer may vary in length depending on the particular conditions and requirement of the application. For example, in the method of the present invention, the nucleotide or oligonucleotide primer is typically 1-24 or more nucleotides in length. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequences has sufficient complementarity with the sequence of the desired template strand to functionally provide a primer-template complex for the synthesis of the extension product.

The terms “RNA synthesis” and “transcription” are used interchangeably and are defined by the specific steps taken by an RNA polymerase of: recognizing and binding to a template initiation site; priming by incorporating a first complementary nucleotide; and adding consecutively complementary nucleotides to elongate the nascent RNA chain.

The term “tag”, “tag sequence” or “protein tag” refers to a chemical moiety, either a nucleotide, oligonucleotide, polynucleotide or an amino acid, peptide or protein or other chemical, that when added to another sequence, provides additional utility or confers useful properties, particularly in the detection or isolation, to that sequence. Thus, for example, a homopolymer nucleic acid sequence or a nucleic acid sequence complementary to a capture oligonucleotide may be added to a primer or probe sequence to facilitate the subsequent isolation of an extension product or hybridized product. In the case of protein tags, histidine residues (e.g., 4 to 8 consecutive histidine residues) may be added to either the amino- or carboxy-terminus of a protein to facilitate protein isolation by chelating metal chromatography. Alternatively, amino acid sequences, peptides, proteins or fusion partners representing epitopes or binding determinants reactive with specific antibody molecules or other molecules (e.g., flag epitope, c-myc epitope, transmembrane epitope of the influenza A virus hemaglutinin protein, protein A, cellulose binding domain, calmodulin binding protein, maltose binding protein, chitin biding domain, glutathione S-transferase, and the like) may be added to proteins to facilitate protein isolation by procedures such as affinity or immunoaffinity chromatography. Chemical tag moieties include such molecules as biotin, which may be added to either nucleic acids or proteins and facilitates isolation or detection by interaction with avidin or streptavidin receptor moiety, and the like. Numerous other tag moieties are known to, and can be envisioned by the trained artisan, and are contemplated to be within the scope of this definition.

The term “template” refers to an oligonucleotide of DNA, or preferably RNA, that serves as one of the substrate for a polymerase. The sequence of a template is complementary to the sequence produced by the polymerase during transcription.

Different “variants” of the HCV NS5B polypeptide exist in nature. These variants may be alleles characterized by differences in the nucleotide sequences of the gene coding for the protein, or may involve different RNA processing or post-translational modifications. The skilled person can produce variants having single or multiple amino acid substitutions, deletions, additions or replacements. These variants may include inter alia: (a) variants in which one or more amino acids residues are substituted with conservative or non-conservative amino acids, (b) variants in which one or more amino acids are added to the HCV NS5B polypeptide, (c) variants in which one or more amino acids include a substituent group, and (d) variants in which the HCV NS5B polypeptide is fused with another peptide or polypeptide such as a fusion partner, a protein tag or other chemical moiety, that may confer useful properties to the HCV NS5B polypeptide, such as, for example, an epitope for an antibody, a polyhistidine sequence, a biotin moiety and the like. Other HCV NS5B polypeptides of the invention include variants in which amino acid residues from one species are substituted for the corresponding residue in another species, either at the conserved or non-conserved positions. In another embodiment, amino acid residues at non-conserved positions are substituted with conservative or non-conservative residues. The techniques for obtaining these variants, including genetic (suppressions, deletions, mutations, etc.), chemical, and enzymatic techniques are known to the person having ordinary skill in the art. To the extent such allelic variations, analogues, fragments, derivatives, mutants, and modifications, including alternative nucleic acid processing forms and alternative post-translational modification forms result in derivatives of the HCV NS5B polypeptide that retain any of the biological properties of the HCV NS5B polypeptide, they are included within the scope of this invention.

The term “vector” as used herein refers to a nucleic acid compound used for introducing exogenous DNA into host cells. A vector comprises a nucleotide sequence which can encode one or more protein molecules. Plasmids, cosmids, viruses, and bacteriophages, in the natural state or which have undergone recombinant engineering, are examples of commonly used vectors, to which another genetic sequence or element (either DNA or RNA) may be attached so as to bring about the replication of the attached sequence or element.

Preferred Embodiments

The invention provides enzymes, methods, assays, and kits for the determination of activity of an HCV RNA-dependent RNA polymerase in the presence and absence of test compounds.

1—Decreased-Affinity NS5B Polymerase Assay

In accordance with a first aspect of the first embodiment of the present invention, there is provided a method for identifying a potential inhibitor of the binding between a HCV NS5B RNA-dependent RNA polymerase and an appropriate primer-template, the method comprising:

a) providing a HCV NS5B polymerase, an appropriate primer-template, and a plurality of appropriate ribonucleotide triphosphates, wherein the HCV NS5B polymerase has an affinity for the primer-template that is decreased relative to that of full length native HCV NS5B RNA-dependent RNA polymerase;

b) incubating the decreased-affinity HCV NS5B polymerase with the primer-template in the presence and absence of a potential inhibitor,

c) measuring the presence of any polymerase products formed upon binding of the decreased-affinity HCV NS5B polymerase and subsequent elongation of the primer upon incorporation of one or more ribonucleotide triphosphates as ribonucleotide monophosphates into the primer in the presence and absence of the potential inhibitor; and

d) comparing the amount of the polymerase products formed in the presence and absence of the potential inhibitor;

wherein a decrease in the amount of the polymerase products formed in the presence of the potential inhibitor compared to the amount of polymerase products formed in the absence of the potential inhibitor is indicative of a potential primer-template binding inhibitor of HCV NS5B RNA-dependent RNA polymerase.

In accordance with a second aspect of the first embodiment, there is provided a method for identifying an inhibitor of HCV NS5B RNA-dependent RNA polymerase, the method comprising the following steps:

carrying out steps a) to d) as described herein;

e) providing a native HCV NS5B RNA-dependent RNA polymerase, an appropriate primer-template, and a plurality of appropriate ribonucleotide triphosphates;

f) incubating the HCV NS5B RNA-dependent RNA polymerase with the primer-template in the presence and absence of a potential inhibitor identified at step (d),

g) measuring the presence of any polymerase products formed upon binding of the HCV NS5B RNA-dependent RNA polymerase and subsequent elongation of the primer upon incorporation of one or more ribonucleotide triphosphates as ribonucleotide monophosphates into the primer in the presence and absence of the potential inhibitor; and

h) comparing the amount of the polymerase products formed in the presence and absence of the potential inhibitor; wherein a decrease in the amount of the polymerase products formed in the presence of the potential inhibitor compared to the amount of polymerase products formed in the absence of the potential inhibitor is indicative of a primer-template binding inhibitor of HCV NS5B RNA-dependent RNA polymerase.

Primer-templates for use in the present invention include those that have the ability to bind to the polymerase of the present invention. In a preferred aspect of the first embodiment, the primer-template comprises a homopolymeric primer-template. In a preferred aspect, the homopolymeric primer comprises a 12 nucleotide RNA oligo-uridylate (or oligo-uridine-monophosphate) (oligo-U) primer and the template comprises a complementary poly-adenylate (or adenosine monophosphate) (polyA) template of heterogeneous length. In a preferred aspect, the template is of 100-10000 nucleotides in length. In alternative aspects of this embodiment, the primer comprises oligo-uridylate of shorter lengths such as U₄, U₆, U₈, U₁₀, or longer lengths such as U₁₄, U₁₆ and U₁₈. In another alternative aspect of this embodiment, different combinations of primer-template with different ratios between the template and the primer may be employed. In another alternative aspect of the present invention, the primer is modified with an affinity tag such as biotin at the free 5'C position to assist in detection of any polymerase products formed in the presence and absence of a test compound.

Ribonucleotide triphosphates for use in the present invention include those that are unlabeled as well as those that are labeled. In a preferred aspect of this first embodiment, the ribonucleotide triphosphates comprise radio-labeled ribonucleotide triphosphates. In a preferred aspect, the ribonucleotide triphosphates comprise UTP-[5,6 ³H] when a 12 nucleotide RNA oligo-uridylate (or oligo-uridine-monophosphate) (oligo-U) primer and a complementary poly-adenylate (or adenosine monophosphate) (polyA) template of heterogeneous length (1000-10000 nucleotides) is used in the assay.

Inhibitors with appropriate selectivity and activity against the NS5B polymerase of the present invention can be identified using the methods and kits of the present invention. In a preferred aspect, test compounds with potential inhibitory activity are of the class of compounds previously identified in WO 02/04425. In a preferred aspect of the present invention, test compounds can include but are not limited to peptides, members of random peptide libraries, combinatorial chemistry-derived molecular libraries, antibodies, carbohydrates, nucleosides, nucleotides or parts thereof, and small organic and inorganic molecules. Test compounds may be endogenous physiological compounds, or natural or synthetic compounds. Test compounds may be one or more discrete compounds from one or more combinatorial libraries. Such libraries can comprise many structurally distinct molecular species. Combinatorial libraries can be used to identify lead compounds or to optimize previously identified leads. Once “lead” compounds are identified using the screening method of the invention, combinatorial chemistry and computational methods can be used to optimize the initial leads. The optimized analogs/variants can be tested in the same screening method that identified the initial lead or in assays using the native NS5B polymerase. Such libraries can be manufactured by well-known methods of combinatorial chemistry and screened by the present method. A potential inhibitor is capable of reducing the biological function of the HCV NS5B polymerase. Preferably, the potential inhibitor reduced or blocks the ability of the polymerase to bind to the primer-template.

In a preferred aspect of the first embodiment, the reaction is incubated at room temperature for 1.5 hours. In alternative aspects of the embodiment, incubation time and temperature can be larger or smaller depending upon the activity of the polymerase at different temperatures.

In a preferred aspect of the first embodiment, concentrations of the primer-template and the nucleotides are lower or near their Kₘ during the screening method to maximize the possibility to detect inhibitors of the reaction.

In a preferred aspect of the first embodiment, test compounds are dissolved in a suitable solvent that ensures the compounds remain in solution during the screening method. In one preferred aspect, an assay buffer containing a final DMSO concentration of 5% is utilized. In an alternative aspect of this first embodiment, the test compounds are dissolved in other suitable solvents known to one of skill in the art. In other alternative aspects, the test compounds are sufficiently soluble in the assay buffer to obviate the need for a cosolvent.

In a preferred aspect of the first embodiment, the method is carried out in a multi-well plate format, for example, a 96-well plate format. Standard high throughput screening methods generally utilize 96-well (8×12) microtiter plates. Typically, these plates can handle up to 500 microlitres. 384-well plates and higher densities can be utilized for miniaturization of method of the present invention. In other embodiments, other sample formats such as cuvettes, Ependorff tubes and the like may be used with the present invention. In a preferred aspect, the assay method of the present invention is fully automated and includes a robotics platform having a liquid handler to dispense reagents integrated with a robotics arm to move plates. In accordance with a preferred aspect of the first embodiment, the method is preferably a homogeneous (“mix and measure”) procedure. That is, reagents used to generate a measurable signal to quantify polymerase reaction products are directly added to the polymerase reaction mixture at the end of the incubation time. More preferably, the substrates comprise a 12 nucleotide RNA oligo-uridylate (or oligo-uridine-monophosphate) (oligo-U) primer modified with biotin at the free 5'C position; a complementary poly-adenylate (or adenosine monophosphate) (polyA) template of heterogeneous length (100-10000 nucleotides); and UTP-[5,6 ³H], and polymerase activity is measured as the incorporation of UMP-[5,6 ³H] into the chain elongated from the oligo-U primer. The ³H-labelled reaction product is captured by scintillation proximity assay (SPA)-beads coated with streptavidin (Amersham-Pharmacia, Biotech, USA) and quantified on the TopCount following procedures that are well known in the art. Based on the results at different concentrations of test compound, standard concentration-% inhibition curves are plotted and analysed to determine IC₅₀'s for the test compounds. In an alternative aspect of the first embodiment, aliquots of reaction mixture are removed at specific reaction times (typically ranging from 15 to 90 min), and bound radioactivity quantitated by liquid scintillation counting for kinetic analysis.

2—Decreased Affinity NS5B Polymerase

In a preferred embodiment of the second embodiment, the NS5B polymerase comprises a hexa-histidine tag fused to the amino terminal portion of the native NS5B. In a preferred embodiment, the decreased-affinity polymerase has a Kₘ for the primer-template of 10 nM or above. In another embodiment, the decreased-affinity polymerase encompasses an RNA-dependent RNA polymerase having a Kₘ value for the primer-template of about 20 nM or above, preferably 60 nM or above, more preferably 100 nM, most preferably 200 nM. According to the second embodiment of this invention, the polymerase for use in the present invention is one that has a decreased affinity for a primer template relative to that of full length native HCV NS5B RNA-dependent RNA polymerase (SEQ ID NO 5). In a preferred aspect of the embodiments of the present invention, the decreased-affinity polymerase encompasses a recombinant HCV NS5B polymerase construct. In a preferred embodiment, the construct comprises an N-terminal hexa-histidine tag full-length HCV NS5B (HT-NS5B; SEQ ID NO 1 or HT-NSA5B; SEQ ID NO 6). In other preferred embodiments, the construct comprises a soluble form of mature HCV NS5B that lacks the C-terminal 21 amino acids and has an N-terminal hexa-histidine or a C-terminal hexa-histidine tag (HT-NS5BΔ21C and NS5BΔ21C-HT; SEQ ID NO 2 and SEQ ID NO 3). In other embodiments, the polymerase comprises a soluble form of mature HCV NS5B that lacks the C-terminal 57 amino acids normally found on the mature NS5B and with a C-terminal hexahistidine tag (NS5BΔ57-HT; SEQ ID NO 4).

3—Kits

In accordance with a third embodiment of the present invention, there is provided a kit for identifying a test compound as a potential inhibitor of the binding between an HCV NS5B polymerase and an appropriate primer-template, the kit comprising:

(a) a first reagent comprising an HCV NS5B polymerase, wherein the HCV NS5B polymerase has an affinity for the primer-template that is decreased relative to that of a full-length native HCV NS5B polymerase;

(b) a second reagent comprising an appropriate primer-template capable of being bound by the HCV NS5B polymerase in the absence of the test compound, wherein the primer is biotinylated at its 5'C position;

(c) a third reagent comprising a plurality of appropriate radio-labeled [5,6 ³H]-ribonucleotide triphosphates capable of being incorporated as radio-labeled ribonucleotide monophosphates into the primer upon binding of the HCV NS5B polymerase and subsequent elongation of the primer, thereby forming polymerase products; and

(d) a fourth reagent comprising a plurality of streptavidin-coated beads containing scintillant suitable to capture the biotinylated primer-template and any formed biotinylated polymerase products, whereby, upon stimulation of the beads, the intensity of light emitted from the beads is proportional to the level of formation of radio-labeled polymerase products.

Preferred aspects of the decreased-affinity HCV NS5B polymerase, appropriate primer-templates, affinity tags, appropriate radio-labeled ribonucleotide triphosphates, and receptor-coated solid support are set out herein.

4—Inhibition of HCV NS5B RNA-Dependent RNA Polymerase

In accordance with a fourth embodiment of the present invention, there is provided a method for inhibiting native HCV NS5B RNA-dependent RNA polymerase comprising the steps of:

(a) identifying an inhibitor of native HCV NS5B RNA-dependent RNA polymerase by the method described herein; and

(b) contacting the native HCV NS5B RNA-dependent RNA polymerase with the test compound identified as having potential inhibitory activity in step (a), whereby polymerase activity of the native HCV NS5B RNA-dependent RNA polymerase is inhibited.

Further details of the preferred embodiments of the invention are illustrated in the following examples which are understood to be non-limiting with respect to the appended claims.

### EXAMPLES

### Example 1

Purification of the NS5B Polymerase Enzymes

HT-NS5B: The NS5B polymerase was produced as a hexa-histidine tagged precursor in Sf-21 insect cells infected from a recombinant baculovirus construct (BacHTa5B). This vector encodes a N-terminal hexa-histidine tag linked to the full-length HCV NS5B (termed HT-NS5B; SEQ ID NO 1). In summary, BacHTa5B infected Sf-21 cell pellets were resuspended in lysis buffer (25 mM Tris pH7.5, 1 mM EDTA, 5 mM MgCl₂, 2 mM β-mercaptoethanol, 500 mM NaCl, 50% glycerol, 0.1% NP-40, 0.05% Triton X-100 and a cocktail of protease inhibitors), Dounce homogenized, treated with DNasel, sonicated and then clarified by centrifugation (105 000×g, 45 min., 4° C.). The resulting supernatant was diluted with 3 volumes of buffer A (25 mM Tris pH7.5, 2 mM β-mercaptoethanol, 10% glycerol, 10 mM imidazole, 500 mM NaCl, 0.1% NP-40, 0.05% Triton X-100 and a cocktail of protease inhibitors) and applied to a Ni-NTA chelating resin (Qiagen). The HT-NS5B protein was eluted by a linear (10-500 mM) imidazole gradient in buffer A, and then diluted with buffer B (20 mM Tris pH 7.5, 20% glycerol, 2 mM β-mercaptoethanol, 1 mM EDTA, 0.1% NP-40 and 0.05% Triton X-100) to reduce the NaCl concentration to 300 mM. The HT-NS5B was applied to a DEAE Sepharose column, to remove nucleic acids and the flow-through was diluted two-fold with buffer B to further reduce the NaCl concentration to 150 mM for the subsequent Hi-trap heparin chromatography. Purified HT-NS5B was eluted with a 200-1000 mM NaCl gradient from the Hi-trap heparin column and stored at —80° C. until use.

HT-NS5BΔ21 or NS5BΔ21-HT: The recombinant HCV NS5B polymerase can be produced in soluble form by expression of a variant that lacks the C-terminal 21 amino acids normally found on the mature NS5B. We have expressed this so called NS5BΔ21 with an N-terminal hexa-histidine (termed HT-NS5BΔ21; SEQ ID NO 2) and with a C-terminal hexa-histidine tag (termed NS5BΔ21-HT; SEQ ID NO 3). Expression of these genes from pET vectors in E. coli strain JM109 (DE3) is induced with 0.4 mM IPTG for 3 hours at 24° C. Cells are harvested and lysed in a microfluidizer in lysis buffer (Tris-HCl pH 7.5, 10% glycerol, 1 mM EDTA, 2 mM 2-mercaptoethanol, 500 mM NaCl, 1 mM PMSF, 1 μg/ml antipain, 1 μg/ml pepstatin A and 1 μg/ml leupeptin). The lysate is clarified by a 30 000 g centrifugation and then supplemented with imidazole to final concentration of 10 mM. The lysate is then loaded onto a metal-chelating resin (Ni-NTA; Qiagen) previously equilibrated with buffer C(25 mM Tris-HCl pH 7.5, 10% glycerol, 1 mM EDTA, 2 mM 2-mercaptoethanol, 500 mM NaCl, 10 mM imidazole, protease inhibitors), washed extensively and then the protein is eluted using a 10 to 500 mM imidazole gradient in buffer C. Peak fractions containing the his-tag NS5BΔ21 are pooled and diluted with buffer D(20 mM Tris-HCl pH 7.5, 10% glycerol, 5 mM DTT) to reduce the NaCl concentration to 300 mM and then applied to a DEAE-Sepharose column to remove any nucleic acid. The flow-through from the DEAE-Sepharose column is diluted with buffer D to reduce the NaCl to 200 mM and then applied to a heparin-Sepharose column. The his-tag NS5B is eluted from the heparin-Sepharose in buffer D with a 200 mM to 1 M NaCl gradient. Peak fractions containing the his-tag NS5B are pooled and diluted with buffer D to achieve a final NaCl of 200 mM and loaded onto a Resource S column. Concentrated his-tag NS5B is eluted from the resource S, loaded and size fractionated on a Superdex 200 column in buffer D containing 300 mM NaCl. Peak fractions contain highly pure his-tag NS5B and are stored at −80 ° C. until use.

NS5BΔ57-HT: The recombinant HCV NS5B polymerase can be produced in soluble form by expression of a variant that lacks the C-terminal 57 amino acids normally found on the mature NS5B. We have expressed this so called NS5BΔ57-HT with a C-terminal hexa-histidine tag (termed NS5BΔ21-HT; SEQ ID NO 4). Expression of these genes from pET vectors in E. coli strain JM109 (DE3) is induced with 0.4 mM IPTG for 3 hours at 24° C. Cells are harvested and lysed in a microfluidizer in lysis buffer (Tris-HCl pH 7.5, 10% glycerol, 1 mM EDTA, 2 mM 2-mercaptoethanol, 500 mM NaCl, 1 mM PMSF, 1 μg/ml antipain, 1 μg/ml pepstatin A and 1 μg/ml leupeptin). The lysate is clarified by a 20 000 g centrifugation and then supplemented with imidazole to final concentration of 15 mM. The lysate is then loaded onto a metal-chelating resin (Ni-NTA; Qiagen) previously equilibrated with buffer E(Tris-HCl pH 7.5, 10% glycerol, 1 mM EDTA, 2 mM 2-mercaptoethanol, 500 mM NaCl, 15 mM imidazole, protease inhibitors), washed extensively and then the protein is eluted using a 15 to 500 mM imidazole gradient in buffer E. Peak fractions containing the his-tag NS5BΔ57 are pooled and diluted with buffer F (25 mM NaPO₄ pH 7.5, 10% glycerol, 2 mM DTT, 1 mM EDTA, 0.1 μg/mL of protease inhibitor cocktail and 0.1 mM PMSF) to reduce the NaCl concentration to 300 mM and then applied to a DEAE-Sepharose column to remove any nucleic acid. The flow-through from the DEAE-Sepharose column is diluted with buffer F to reduce the NaCl to 200 mM and then applied to a heparin-Sepharose column. The HT-NS5B is eluted from the heparin-Sepharose in buffer F with a 200 mM to 1 M NaCl gradient. Peak fractions containing the HT-NS5B are pooled and diluted in buffer G (25 mM NaPO₄ pH 7.5, 10% glycerol, 2 mM DTT) to achieve a final NaCl concentration of 140 mM and loaded onto a Q-Sepharose column equilibrated in buffer G containing 140 mM NaCl. The flow-through from the Q-Sepharose column is collected and adjusted with 5 M NaCl to a final concentration of 300 mM NaCl. The HT-NS5B eluted in the flow-through is then concentrated with Centrifugal concentrators and stored at −80° C. until use.

Full length native NS5B (SEQ ID NO 5): Is produced as a histidine-tagged precursor (HT-NSA5B; SEQ ID NO 6) from a recombinant baculovirus as described above for the HT-NS5B. This precursor contains a NS5A-NS5B cleavage site for the NS3 protease that permits the removal of heterologous sequences at the amino terminus of the NS5B by the NS3/4A protease. The NS3/4A protease cleaves the NS5A-5B cleavage site to generate the mature NS5B and uses a 1:50:1.25 molar ratio of NS3 protease:4A cofactor peptide:HT-NSA5B precursor in buffer H (20 mM Tris pH 7.5, 20% glycerol, 2 mM β-mercaptoethanol, 1 mM EDTA, 0.15% n-dodecyl-β-D-maltoside) diluted with an equal volume of buffer 1(50 mM NaPO₄ pH 7.8, 10% glycerol, 0.3 M NaCl, 0.1% n-dodecyl-β-D-maltoside). The reaction is performed at room temperature for 45 min., followed by a 5 hour incubation at 4° C. Following NS3 catalyzed removal of the His-tag-5A, the reaction mixture is supplemented with 10 mM imidazole and batch-mixed with Ni-NTA resin to bind the cleaved His-tag tails and any uncleaved HT-NS5B protein. The resin is pelleted by centrifugation and the supernatant (mature NS5B fraction termed NS5B; SEQ ID NO 5) is subjected to Hi-trap heparin chromatography as described above to separate the NS3 protease from NS5B RdRp. The NS5B fractionated by heparin chromatography is applied to a preparative Superose-12 gel filtration column, in buffer H containing 800 mM NaCl, to recover a highly pure NS5B.

### Example 2

Inhibition of Native NS5B RNA-Dependent RNA Polymerase Acivity

Compounds such as those chosen from compounds as set out in WO 02/04425 were tested for inhibitory activity against the hepatitis C virus RNA-dependent RNA polymerase (NS5B), according to the following assay:

The substrates were:

a 12 nucleotide RNA oligo-uridylate (or oligo-uridine-monophosphate) (oligo-U) primer modified with biotin at the free 5'C position;

a complementary poly-adenylate (or adenosine monophosphate) (polyA) template of heterogeneous length (100-10000 nucleotides); and

UTP-[5,6 ³H].

Polymerase activity was measured as the incorporation of UMP-[5,6 ³H] into the chain elongated from the oligo-U primer. The ³H-labelled reaction product was captured by SPA-beads coated with streptavidin (Amersham-Pharmacia Biotech, USA) and quantified on the TopCount following procedures that are well known in the art.

All solutions were made from DEPC-treated MilliQ water (2 ml of DEPC is added to 1L of MilliQ water; the mixture was shaken vigorously to dissolve the DEPC, then autoclaved at 121° C. for 30 minutes).

Enzyme: The full length HCV HT-NS5B (SEQ ID NO 1) was purified as an N-terminal hexa-histidine fusion protein as described above in Example 1. The enzyme can be stored at −20° C. in storage buffer. Under these conditions, it was found to maintain activity for at least 6 months.

Substrates: The biotinylated oligo-U₁₂ primer, the Poly(A) template, and the UTP-[5,6 ³ H] were dissolved in water. The solutions can be stored at −80° C.

| | | |
|---|---|---|
| Assay buffer: | 20 | mM Tris-HCl pH 7.5 |
| | 5 | mM MgCl₂ |
| | 1 | mM EDTA |
| | 1 | mM DTT |
| NS5B storage buffer: | 0.1 | μM NS5B |
| | 25 | mM Tris-HCl pH 7.5 |
| | 300 | mM NaCl |
| | 5 | mM DTT |
| | 1 | mM EDTA |
| | 0.1% | n-Dodecyl maltoside |
| | 30% | glycerol |

Test compound cocktail: Just prior to assay, test compounds were dissolved in assay buffer containing 15% DMSO.

Substrate cocktail: Just prior to assay, the substrates were mixed in assay buffer to the following concentrations:

| | Concentration in | | Final | |
|---|---|---|---|---|
| Component | substrate cocktail | | Concentration in assay | |
| RNAsin ™ | 0.5 | U/μl | 1.67 | U/μl |
| Biotin-oligo-U₁₂ primer | 3 | ng/μl | 1 | ng/μl |
| PolyA template | 30 | ng/μl | 10 | ng/μl |
| UTP-[5,6-³H] | 0.025 | μCi/μl | 0.0083 | μCi/μl |
| 35 Ci/mmol | | | 0.25 | μM |
| UTP | 2.25 | μM | 0.75 | μM |

Enzyme cocktail: Just prior to assay, the RNA polymerase (NS5B) cocktail was prepared in assay buffer to the following specifications:

| Component | Concentration in cocktail |
|---|---|
| Tris-HCl at pH 7.5 | 20 mM |
| MgCl₂ | 5 mM |
| EDTA | 1 mM |
| DTT | 1 mM |
| n-Dodecyl maltoside | 1% |
| NS5B | 30 nM |

Protocol:

The assay reaction was performed in a Microfluor™ white “U” bottom plate (Dynatech™ #7105), by successively adding:

20 μl of test compound cocktail;

20 μl of substrate cocktail; and

20 μl of enzyme cocktail

(final [NS5B] in assay=10 nM; final [n-dodecyl maltoside] in assay=0.33%; final DMSO in assay=5%).

The reaction was incubated at room temperature for 1.5 hours. STOP solution (20 μl; 0.5 M EDTA, 150 ng/μl tRNA) was added, followed by 30 μl streptavidin coated PVT beads (8 mg/ml in 20 mM Tris-HCl, pH 7.5,25 mM KCl, 0.025% NaN₃). The plate was then shaken for 30 minutes. A solution of CsCl was added (75 μl, 5 M), to bring the CsCl concentration to 1.2 M. The mixture was then allowed to stand for 1 hour. The beads were then counted on a Hewlett Packard TopCount™ instrument using the following protocol:

Data mode: counts per minute

Scintillator: liq/plast

Energy range: low

Efficiency mode: normal

Region: 0-50

Count delay: 5 Minutes

Count time: 1 minute

Expected results: 6000 cpm/well

200 cpm/well no enzyme control

Based on the results at ten different concentrations of test compound, standard concentration-% inhibition curves were plotted and analysed to determine IC₅₀'s for the test compounds. For some compounds, the IC₅₀ was estimated from two points.

### Example 3

Evaluation of the Effect of Primer Length on the Polymerase Activity With HT-NS5B

Studies examining the optimal homopolymeric primer-template for the NS5B polymerase reaction have been conducted in the prior art. Different combinations of substrate in addition to different ratios between the template and the primer have been tested. It has been previously shown that polyG/oligoC and polyU/oligoA were not efficiently used by the enzyme (Lohmann et al., 1997; Oh et al., 1999; Johnson et al, 2000), whereas polyC/oligoG and polyA/oligoU were efficient templates/primers (Lohmann et al., 1997; Lohmann et al., 1998; Ferrari et al.,1999; Oh et al., 1999; Ishii et al., 1999; Tomei et al, 2000; Johnson et al, 2000). Different lengths of oligo primers have been used in the polymerase reaction (usually from 12 to 20-mers).

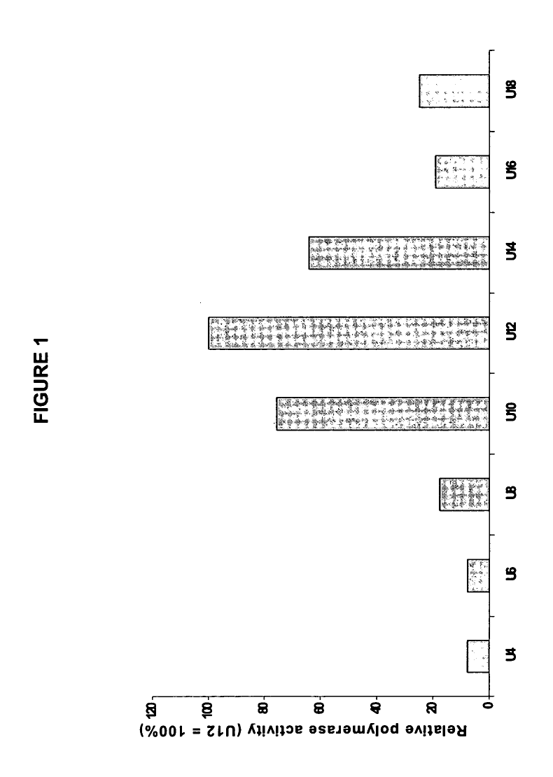

The effect of primer length on HT-NS5B enzymatic activity was studied using Oligos U₄, U₆, U₈, U10, U₁₂, U₁₄, U₁₆ and U₁₈ (Genset SA, Paris, France). Their respective concentrations were determined by OD absorption at 260 nm and then adjusted to obtain 500 nM final concentration of each oligo in the assay. Poly(A) was maintained at 20 μg/mL final concentration. Conditions of all other reagents in the reaction were similar to those as described in Example 4 for the Kₘ determination protocol. Final concentrations of the HT-NS5B enzyme and of UTP used in the assay were of 15 nM and 1 μM respectively. Velocities of the reactions were determined for the different oligos and compared to the standard reaction in which U₁₂ was used. The Results are illustrated in FIG. 1. As indicated in FIG. 1, the maximal rate of nucleotide incorporation was obtained with U₁₂. Reactions reconstituted with oligos shorter or longer than U₁₂ showed a decreased velocity of incorporation.

### Example 4

Kinetic Analyses of Different Constructs of NS5B Polymerases

Assays with recombinant HCV polymerase in vitro reported in the prior art use different templates and primers, different truncated and tagged polymerase constructs, and because of the complexity of performing steady-state kinetics with this multi-substrate system, enzymology studies have reported significantly different Kₘ values both for primer-template and NTP. Most of these studies were directed toward the Kₘ determination of the nucleotide triphosphate implicated in the reaction. Using polyC/oligoG as primer-template, the reported Kₘ for GTP varies depending on the study performed. Values between 0.2 μM and 52 μM (Tomei et al, 2000; Lohmann et al., 1998; Ferrari et al., 1999) have been obtained. With polyA/oligoU, the Kₘ for UTP also varies. Values between 5 μM and 22 μM (Tomei et al, 2000; Johnson et al, 2000; Lohmann et al., 1998) have been obtained. Regarding Kₘ determination for primer-template, a high affinity value was reported between polyC/oligoG and the HCV polymerase (Kₘ of 30 nM) (Ferrari et al., 1999).

In a polymerase reaction with multiple substrates the reaction presumably follows a sequential order: the polymerase first binds to the primer-template to form a binary complex which then binds a nucleotide to form the catalytically competent ternary complex.

To determine the kinetic parameters for one substrate, the enzyme must be saturated by the other. The initial velocities at increasing concentrations of each substrate were determined. The data were processed and analyzed with kinetics software (GraFit Erithacus Software). To estimate the Kₘ for the primer-template (polyA/oligoU₁₂), a saturating amount of UTP (25 to 50 μM) was used in the assay in presence of increasing concentration of polyA/oligoU₁₂ (10 nM to 1000 nM).

Similarly, in the determination of the Kₘ for UTP, a saturating amount of primer-template was used (400 to 1000 nM) in the presence of increasing concentrations of UTP.

We have examined a variety of purified HCV NS5B polymerases, incorporating the features such as affinity tags and C-terminal truncations as described in Example 1, and characterized the activity of these different NS5B variants. The activity of NS5B in a common in vitro polymerase reaction with homopolymeric RNA requires interaction with multiple substrates that include a primer-template and a ribonucleotide triphosphate.

Steady-state kinetic parameters, such as the Kₘ, can be determined for both the primer-template and the ribonucleotide triphosphate substrates (Ferrari et al., 1999).

Polymerase reactions were performed in 10 mM Tris pH 7.5, 1 mM EDTA, 5 mM MgCl₂, 0.17 units/μL RNasin, 0.33% dodecyl-β-D-maltoside, 3% glycerol, 0.01% Igepal and 30 mM NaCl. In the determination of the Kₘ of the different enzyme constructs towards poly(A)/oligoU₁₂, final concentrations of the primer in the assay ranged from 10 nM to 1000 nM (a primer-template ratio of 10 was maintained at all concentrations) and final UTP concentration was 25 or 50 μM (containing 0.08 to 0.2 μCi/μL of ³³P-UTP). Primer-template and polymerase were mixed, before addition of UTP to trigger the reaction. Aliquots of 8 μL were removed at specific times (ranging from 15 to 90 min), spotted onto DE81 filter paper discs, and dried completely. The discs were then washed three times for 10 minutes with 1 M sodium phosphate pH7, and then rinsed in water and 85% ethanol. Bound radioactivity was then quantitated by liquid scintillation counting in 5 mL of Optiphase ‘HiSafe’ 2. The initial velocity at each concentration of poly(A)/oligoU₁₂ was determined. Data were processed and analyzed with kinetics software (GraFit Erithacus Software) to obtain the Kₘ for poly(A)/oligoU. The same procedure was applied to determine the Kₘ for UTP (in presence of saturating amount of poly(A)/oligoU₁₂ (from 400 nM to 1000 nM).

The different NS5B polymerase constructs (NS5B, HT-NS5B, HT-NS5BΔ21C, NS5BΔ21C-HT, and NS5BΔ57-HT) were produced and purified as described above in Example 1. The Kₘ values of both UTP and polyA/oligoU for each of these enzyme constructs were determined. The results illustrated in Table I below show that the Kₘ for UTP varies from 0.8 μM to 8 μM, depending of the polymerase construct. However, the Kₘ for the primer-template fluctuated by approximately 30-fold amongst the different polymerase constructs, with values ranging from 7 nM to 200 nM.

TABLE 1
Kinetic Analyses of different constructs of NS5B polymerases

| | | HT- | NS5BΔ57- | | |
|---|---|---|---|---|---|
| | HT-NS5B | NS5BΔ21 | HT | NS5BΔ21-HT | NS5B |
| Kₘ (P/T) | 0.20 | 0.06 | 0.021 | 0.013 | 0.007 |
| (μM) | | | | | |
| Kₘ (UTP) | 5.0 | 8.2 | 0.8 | 1.5 | 6.0 |
| (μM) | | | | | |

### Example 5

Determination of the Kᵢ and of the Mode of Inhibition for Test Compounds.

A class of inhibitors of the NS5B polymerase were previously identified in WO 02/04425 using the method of the present invention. In order to determine the mode of inhibition with a representative compound from this class of inhibitors, two series of reactions were performed.

In the first series, HT-NS5B (see Example 1) polymerase reactions were performed by examining reaction velocity at different primer-template and inhibitor concentrations. The concentration of primer-template ranged from 25 and 1000 nM with a fixed concentration of UTP at 25 μM (containing up to 0.2 μCi ³³P-UTP/μL). The concentration of enzyme used in the assay was 5 nM and the concentration of inhibitor ranged from 0.25 to 4-fold the IC₅₀ value. For each of these incubations, the velocity of the reaction was determined by withdrawing aliquots at defined times and transferring them on DE81 filter discs and dried completely. The discs were then washed three times for 10 minutes with 1 M sodium phosphate pH7, and then rinsed in water and 85% ethanol. Bound radioactivity was then quantified by liquid scintillation counting in 5 mL of Optiphase ‘HiSafe’ 2. The initial velocity at each concentration of poly(A)/oligoU₁₂ and inhibitor was determined. Data were processed as described in Example 4.

In the second series of experiments, velocity of the reaction was monitored at different UTP and inhibitor concentrations. The primer-template concentration was fixed (at approximately 250 nM), the UTP concentration ranged from 0.25 μM to 50 or 100 μM (with 0.02 to 0.2 μCi ³³P-UTP/μL), and the concentration of the inhibitor ranged from 0.25 to 4-fold the IC₅₀ value with the enzyme concentration between 10 and 25 nM.

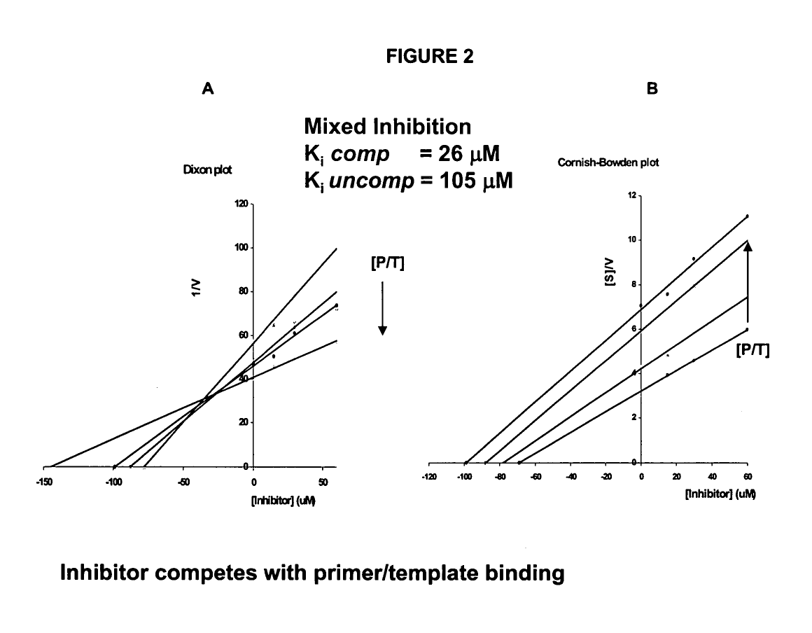

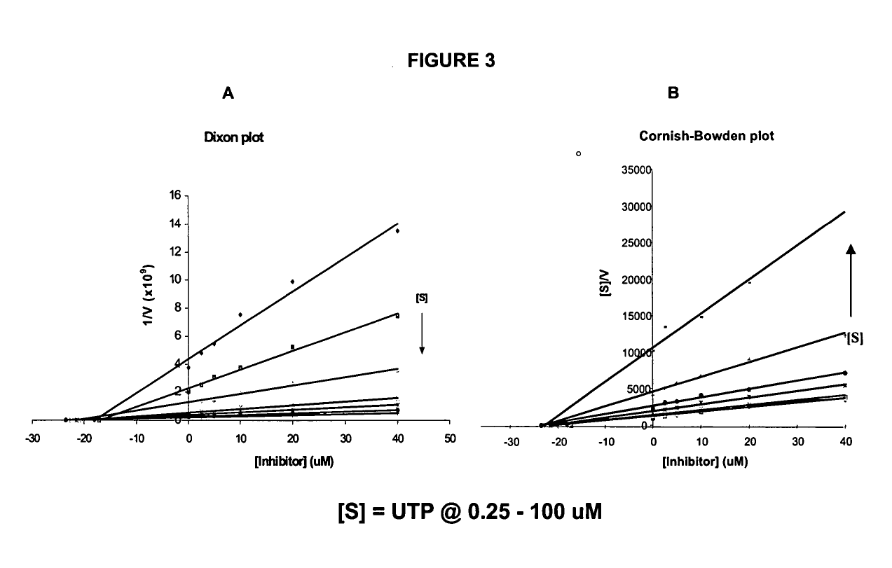

Kinetic results were then plotted according to the method of Cornish-Bowden (1974), allowing for determination of the mode of inhibition as well as the constant(s) of inhibition (Kᵢ as the competitive part of inhibition and Kᵢ′ as the uncompetitive part of inhibition). Examples of these graphs are shown in FIGS. 2 and 3. A mixed-mode of inhibition was observed towards the primer-template, with a major competitive component (Intercept of the Dixon plot over the x axis and intercept of the Cornish-Bowden plot below the x axis).

In contrast, a clear non-competitive inhibition of UTP was obtained, as both Dixon and Cornish-Bowden plots intercept on the x axis.

### Example 6

Variations in IC₅₀ Values Obtained With Different Polymerase Constructs.

The IC₅₀ values for a series of 30 related compounds from the same class of compounds as the test compounds in Example 5 were determined with the five different constructs of the NS5B polymerase as described in Example 1. Results of these experiments are illustrated in Table 2 below.

TABLE 2
Determination of IC₅₀ values for polymerase constructs

| | Fold-increase in the IC₅₀ values |
|---|---|
| Polymerase | relative to HT-NS5B |
| HT-NS5B | 1 |
| HT-NS5BΔ21 | 4.2 |
| NS5BΔ57-HT | 42 |
| NS5BΔ21-HT | 56 |
| NS5B | 128 |

As detailed in Example 5, inhibitors of this class were found to be competitive with the primer-template (demonstrated through the mode of inhibition and Kᵢ and Kᵢ′ values as determined in Example 5), the IC₅₀ of this series of inhibitors was explored. Based on the different Kₘ of the various NS5B constructs towards the primer-template, the IC₅₀ of this series of inhibitors was predicted to also differ with the various NS5B constructs. Table 2 summarizes the observed results. The HT-NS5B enzyme used in Example 1 was considered as the reference value (normalized to 1), such that increases in IC₅₀'s for this series of compounds varied from 4.2-fold with the HT-NS5BΔ21 enzyme up to 128-fold with the full length native NS5B. The dramatic increases in IC₅₀ values indicates that screening for inhibitors of primer-template binding with the full length native NS5B would have greatly impaired identification of moderately potent compounds.

All publications, patents and patent applications are herein incorporated by reference in their entirety to the same extent as if each individual publication, patent or patent application was specifically and individually indicated to be incorporated by reference in its entirety.

References:

Ago et al. 1999, Structure 7: 1417-1426

Ausubel et al., 1994, Current Protocols in Molecular Biology, Wiley, New York.

Bartenschlager, R. et al., 1993, J. Virol., 67, 3835-3844.

Bartenschlager, R. et al., 1994, J. Virol., 68, 8147-8157.

Bartenschlager, R. 1999, J Viral Hepat., 6, 165-81.

Behrens et al., 1996, Embo J.,, 15, 12-22.

Bressanelli et al. 1999, Proc. Natl. Acad. Sci, USA 96: 13034-13039

Blight et al. 2000, Science 290: 1972-1974

Cho et al., 1998, J. Biol. Chem., 273, 15045

Choo et al., 1989, Science 244, 359-362.

Cornish-Bowden A., 1974, Biochem. J., 137, 143-144.

Dash et al., 1997, Am. J. Pathol., 151, 363-373

Ferrari et al., 1999,J. Virol.,, 73, 1649-1654.

Fourner et al. 1998, J. Gen. Virol. 79, 2376

Gale Jr. et al. 1997 Virology 230, 217

Grakoui, A. et al., 1993(a), J. Virol. 67, 1385-1395.

Grakoui A, et al., 1993(b), Proc Natl Acad Sci USA, 90, 10583-7

Guo et al. (2001) J. Virol. 8516-8523

Hijikata, M. et al., 1991, Proc. Natl. Acad. Sci. USA. 88, 5547-5551.

Hijikata, M. et al., 1993, J. Virol. 67, 4665-4675.

Hirowatari, Y. et al., 1995, Anal. Biochem., 225, 113-220.

Ikeda et al. 1998, Virus Res. 56, 157

Ishii et al., 1999,Hepatology,, 29, 1227-1235.

Ito et al. 1996, J. Gen. Virol. 77, 1043-1054

IUPAC-IUB Biochemical Nomenclature Commission, 1972, Biochemistry, 11, 1726-1732.

Johnson et al, Arch. Biochem. Biophys., 2000; 377, 129-134.

Kim, D. W. et al., 1995, Biochem. Biophys. Res. Comm., 215, 160-166.

Kim et al., 1996, Cell, 87, 343;

Kim et al., 1998, Structure, 6, 89

Kim et al., 1999, Arch. Virol, 144, 329-343.

Krieger et al. 2001, J. Virol. 4614-4624

Kwong A D. et al., 1998, Antiviral Res., 40, 1-18

Kolykhalov et al., 1996, J. Virol., 70, 3363-3371.

Kolykhalov, A. A. et al., 2000; J. Virol. 74: 2046-2051

Khromykh et al., 1997, J. Virol. 71, 1497

Kwong A D. et al., 1998, Antiviral Res. 40, 1-18.

Lai, M M C, 1998, Virology 244, 1-12.

Lanford et al. 1994, Virology 202, 606

Lesburg et al. 1999, Nat. Struct. Biol. 6: 937-943

Lohmann et al., 1997, J. Virol.,, 71, 8416-8428

Lohmann et al., 1998,Virology, 249, 108-118.

Lohmann et al., 1999, J Biol Chem., 274, 10807-15.

Lohman et al. 1999, Science 285: 110-113

Lohman et al. 2001 J. Virol. 1437-1449

Love, R. A. et al., 1996, Cell, 87, 331-342

Luo et al., 2000, J. Virol. 74(2), 851-863.

Martell et al. 1999 J. Clin. Microbiol. 37: 327-332

Mizutani et al. 1996, J. Virol. 70, 7219-7223

Moser et al., 1998, J. Virol. 72, 5318

Oh et al., 1999, J. Virol. 73(9), 7694-7702.

Oh et al., 2000, J. Biol. Chem. 275(23): 17710-17717, web manuscript M908781199.

Qin et al., 2001, Hepatology, 33, 728-737.

Qin et al., 2002,J. Biol. Chem., 277, 2132-2137.

Reed et al., 1997, J. Virol. 71, 7187

Reed, K. E. and Rice, C. M., 2000, Current Topics in Microbiology & Immunology, 242, 55-84.

Sambrook et al., 1989, Molecular Cloning—A Laboratory Manual, Cold Spring Harbor Labs.

Shimizu et al. 1993, PNAS, USA, 90, 6037-6041

Strauss, J. H., and Strauss, E. G. 1999, Science 283, 802-804

Sun, X. L, et al., 2000, Biochem. Biophys. Res. Commun. 268(3); 798-803.

Tanaka, T. et al., 1996, J. Virol. 70, 3307-3312.

Tomei et al., 2000,J. Gen. Virology, 81, 759-767.

Yamashita, T. et al., 1998, J. Biol. Chem. 273, 15479-15486.

Yanagi et al., 1999, Proc. Natl. Acad. Sci. USA, 96, 2291-95

Yao et al., 1997, Nature Structural Biology, 4, 463

Yem et al., 1998, Protein Science, 7, 837

Yoo et al., 1995, J. Virol., 69, 32-38

Yuan et al., 1997, Biochem. Biophys. Res. Comm., 232, 231-235

Zhong et al., 2000, J. Virol. 74(4), 2017-2022

Although preferred embodiments of the invention have been described herein, it will be understood by those skilled in the art that variations may be made thereto without departing from the spirit of the invention or the scope of the appended claims.

We claim:

1. A method for identifying a potential inhibitor of the binding between a HCV NS5B RNA-dependent RNA polymerase and an appropriate primer-template, the method comprising the following steps:
a) providing a HCV NS5B polymerase, an appropriate primer-template, and a plurality of appropriate ribonucleotide triphosphates, wherein the HCV NS5B polymerase has an affinity for the primer-template that is decreased relative to that of native HCV NS5B RNA-dependent RNA polymerase;
b) incubating the HCV NS5B polymerase with the primer-template in the presence and absence of a potential inhibitor,
c) measuring the presence of any polymerase products formed upon binding of the HCV NS5B polymerase and subsequent elongation of the primer upon incorporation of one or more ribonucleotide triphosphates as ribonucleotide monophosphates into the primer in the presence and absence of the potential inhibitor; and
d) comparing the amount of the polymerase products formed in the presence and absence of the potential inhibitor;
wherein a decrease in the amount of the polymerase products formed in the presence of the potential inhibitor compared to the amount of polymerase products formed in the absence of the potential inhibitor is indicative of a potential primer-template binding inhibitor of HCV NS5B RNA-dependent RNA polymerase.

2. The method according to claim 1, wherein the decreased-affinity NS5B polymerase has a Kₘ value for the primer-template of above 10 nM.

3. The method according to claim 1, wherein the decreased-affinity NS5B polymerase has a Kₘ value for the primer-template of above 60 nM.

4. The method according to claim 1, wherein the decreased-affinity NS5B polymerase has a Kₘ value for the primer-template of about 100 nM or above.

5. The method according to claim 1, wherein the decreased-affinity NS5B polymerase has a Kₘ value for the primer-template of about 200 nM.

6. The method according to claim 1, wherein the decreased-affinity NS5B polymerase is a native NS5B polypeptide with a histidine-tag fused to its N-terminus.

7. The method according to claim 1, wherein the decreased-affinity NS5B polymerase has a sequence according to SEQ ID No.1.

8. The method according to claim 1, wherein the decreased-affinity NS5B polymerase has a sequence according to SEQ ID No.2.

9. The method according to claim 1, wherein the decreased-affinity NS5B polymerase has a sequence according to SEQ ID No.3.

10. The method according to claim 1, wherein the decreased-affinity NS5B polymerase has a sequence according to SEQ ID No.4.

11. The method of claim 1, wherein the template comprises a poly-adenylate template and the primer comprises a RNA oligo-uridylate primer.

12. The method according to claim 1, wherein the primer is biotinylated at the free 5'C position, the ribonucleotide triphosphates comprise radio-labeled [5,6 ³H]-ribonucleotide triphosphates, and the amount of polymerase products in the presence and absence of the potential inhibitor of step (c) is measured by way of a scintillation proximity assay (SPA), whereby a plurality of streptavidin-coated beads containing scintillant are operable to capture the biotinylated primer-template and any formed biotinylated polymerase products, and whereby, upon stimulation of the beads, the intensity of light emitted from the beads is proportional to the level of formation of radio-labeled polymerase products.

13. The method of claim 1, where the incubation temperature is about room temperature.

14. The method of claim 1, wherein the initial concentration of potential inhibitor provided is less than or equal to the Kₘ value that the decreased-affinity NS5B polymerase has for the primer-template.

15. A decreased-affinity NS5B polymerase having a Kₘ value for its primer-template of above 10 nM.

16. A decreased-affinity NS5B polymerase having a Kₘ value for its primer-template of above 60 nM.

17. A NS5B polymerase according to claim 15, wherein the decreased-affinity NS5B polymerase has a Kₘ value for the primer-template of about 100 nM or above.

18. A NS5B polymerase according to claim 15, wherein the decreased-affinity NS5B polymerase has a Kₘ value for the primer-template of about 200 nM.

19. A NS5B polymerase according to claim 15, wherein the decreased-affinity NS5B polymerase is a native NS5B polypeptide with a histidine-tag fused to its N-terminus.

20. A NS5B polymerase according to claim 15, wherein the decreased-affinity NS5B polymerase has a sequence according to SEQ ID No.1.

21. A NS5B polymerase according to claim 15, wherein the decreased-affinity NS5B polymerase has a sequence according to SEQ ID No.2.

22. A NS5B polymerase according to claim 15, wherein the decreased-affinity NS5B polymerase has a sequence according to SEQ ID No.3.

23. A NS5B polymerase according to claim 15, wherein the decreased-affinity NS5B polymerase has a sequence according to SEQ ID No.4.

24. A method for identifying an inhibitor of HCV NS5B RNA-dependent RNA polymerase, the method comprising the following steps:
a) providing a HCV NS5B polymerase, an appropriate primer-template, and a plurality of appropriate ribonucleotide triphosphates, wherein the HCV NS5B polymerase has an affinity for the primer-template that is decreased relative to that of native HCV NS5B RNA-dependent RNA polymerase;
b) incubating the HCV NS5B polymerase with the primer-template in the presence and absence of a potential inhibitor,
c) measuring the presence of any polymerase products formed upon binding of the HCV NS5B polymerase and subsequent elongation of the primer upon incorporation of one or more ribonucleotide triphosphates as ribonucleotide monophosphates into the primer in the presence and absence of the potential inhibitor;
d) comparing the amount of the polymerase products formed in the presence and absence of the potential inhibitor; wherein a decrease in the amount of the polymerase products formed in the presence of the potential inhibitor compared to the amount of polymerase products formed in the absence of the potential inhibitor is indicative of a potential primer-template binding inhibitor of HCV NS5B RNA-dependent RNA polymerase;
e) providing a HCV NS5B RNA-dependent RNA polymerase, an appropriate primer-template, and a plurality of appropriate ribonucleotide triphosphates;
f) incubating the HCV NS5B RNA-dependent RNA polymerase with the primer-template in the presence and absence of a potential inhibitor identified at step (d),
g) measuring the presence of any polymerase products formed upon binding of the HCV NS5B RNA-dependent RNA polymerase and subsequent elongation of the primer upon incorporation of one or more ribonucleotide triphosphates as ribonucleotide monophosphates into the primer in the presence and absence of the potential inhibitor; and
h) comparing the amount of the polymerase products formed in the presence and absence of the potential inhibitor; wherein a decrease in the amount of the polymerase products formed in the presence of the potential inhibitor compared to the amount of polymerase products formed in the absence of the potential inhibitor is indicative of a primer-template binding inhibitor of HCV NS5B RNA-dependent RNA polymerase.

25. A kit for identifying a test compound as an inhibitor of the binding between an HCV NS5B polymerase and an appropriate primer-template, the kit comprising:
(a) a first reagent comprising an HCV NS5B polymerase, wherein the HCV NS5B polymerase has an affinity for the primer-template that is decreased relative to that of a native HCV NS5B polymerase;
(b) a second reagent comprising an appropriate primer-template capable of being bound by the HCV NS5B polymerase in the absence of the test compound, wherein the primer is affinity-tagged;
(c) a third reagent comprising a plurality of appropriate radio-labeled [5,6 ³H]-ribonucleotide triphosphates capable of being incorporated as radio-labeled [5,6 ³H]-ribonucleotide monophosphates into the primer upon binding of the HCV NS5B polymerase and subsequent elongation of the primer, thereby forming polymerase products; and
(d) a fourth reagent comprising a plurality of receptor-coated solid support suitable to capture the affinity-tagged primer-template and any formed affinity-tagged polymerase products, whereby, upon measurement, intensity of signal emitted from the solid support is proportional to the level of formation of radio-labeled polymerase products.

26. A kit for identifying a test compound as an inhibitor of HCV NS5B RNA-dependent RNA polymerase, the kit comprising:
(a) a first reagent comprising an HCV NS5B polymerase, wherein the HCV NS5B polymerase has an affinity for the primer-template that is decreased relative to that of a native HCV NS5B RNA-dependent RNA polymerase;
(b) a second reagent comprising an appropriate primer-template capable of being bound by the decreased-affinity HCV NS5B polymerase in the absence of the test compound, wherein the primer is biotinylated at its 5'C position;
(c) a third reagent comprising a plurality of appropriate radio-labeled [5,6 ³H]-ribonucleotide triphosphates capable of being incorporated as radio-labeled [5,6 ³H]-ribonucleotide monophosphates into the primer upon binding of the decreased-affinity HCV NS5B polymerase and subsequent elongation of the primer, thereby forming polymerase products; and
(d) a fourth reagent comprising a plurality of streptavidin-coated beads containing scintillant suitable to capture the biotinylated primer-template and any formed biotinylated polymerase products, whereby, upon stimulation of the beads, the intensity of light emitted from the beads is proportional to the level of formation of radio-labeled polymerase products by the decreased-affinity HCV NS5B polymerase, whereby a decrease in the of radio-labeled polymerase products is indicative of a primer-template binding inhibitor of HCV NS5B RNA-dependent RNA polymerase.

27. A screening assay for identifying a potential inhibitor of the binding between a HCV NS5B RNA-dependent RNA polymerase and an appropriate primer-template, the assay comprising the following steps:
a) providing a HCV NS5B polymerase, an appropriate primer-template, and a plurality of appropriate ribonucleotide triphosphates, wherein the HCV NS5B polymerase has an affinity for the primer-template that is decreased relative to that of native HCV NS5B RNA-dependent RNA polymerase;
b) incubating the HCV NS5B polymerase with the primer-template in the presence and absence of a potential inhibitor,
c) measuring the presence of any polymerase products formed upon binding of the HCV NS5B polymerase and subsequent elongation of the primer upon incorporation of one or more ribonucleotide triphosphates as ribonucleotide monophosphates into the primer in the presence and absence of the potential inhibitor; and
d) comparing the amount of the polymerase products formed in the presence and absence of the potential inhibitor;
wherein a decrease in the amount of the polymerase products formed in the presence of the potential inhibitor compared to the amount of polymerase products formed in the absence of the potential inhibitor is indicative of a potential primer-template binding inhibitor of HCV NS5B RNA-dependent RNA polymerase.

28. The method according to claim 1, wherein the decreased-affinity NS5B polymerase has a sequence according to SEQ ID No. 6.

29. A NS5B polymerase according to claim 15, wherein the decreased-affinity NS5B polymerase has a sequence according to SEQ ID No. 6.
